(19) United States
(12) Patent Application Publication
(10) Pub. No.: US 2008/0161747 A1
(43) Pub. Date: Jul. 3, 2008

(54) ELECTRICALLY CONDUCTIVE MICRONEEDLE ROLLER

(76) Inventors: JUSTIN LEE, Tustin, CA (US); Soon Il Hong, Si-heung-si (KR)

Correspondence Address: PARK LAW FIRM, 3255 WILSHIRE BLVD, SUITE 1110, LOS ANGELES, CA, 90010, US

(21) Appl. No.: 11/961,060

(22) Filed: Dec. 20, 2007

(30) Foreign Application Priority Data
Dec. 20, 2006 (KR) 2006-0131236

(51) Int. Cl. A61N 1/30

(52) U.S. Cl. 604/20

(57) ABSTRACT

An electrically conductive microneedle roller includes stacked discs, each of which includes a plurality of radial grooves, a plurality of microneedles that are received in the radial grooves of the disc, an electrically conductive bracket that supports the stacked discs, and a handle that supports the bracket. Electric current flows to the skin via the microneedles and provides electric stimulation. The discs are assembled using UV bond thereby reducing the assembly time. The roller has enhanced service life since the microneedles do not fall off from the roller since radial grooves holding the microneedles have tapered shape.

### CLAIMING FOREIGN PRIORITY

The applicant claims and requests a foreign priority, through the Paris Convention for the Protection of Industrial Property, based on patent applications filed in the Republic of Korea (South Korea) with the filing date of Dec. 20, 2006 with the patent application number 10-2006-0131236 by the applicant, the contents of which are incorporated by reference into this disclosure as if fully set forth herein.

### BACKGROUND OF THE INVENTION

The present invention relates to a microneedle roller.

More particularly, the present invention relates to a microneedle roller that can provide electric current to the microneedles.

The skin of a human being is composed of three primary layers, the epidermis, the dermis and the appendages. Among them, the dermis forming skin wrinkles is disposed under the epidermis as the lowest skin layer at a thickness of about 0.7 mm to 4 mm. Collagen in the dermis shrinks as the aging proceeds. As the result, the skin gets dry, and more skin wrinkles appear. In order to prevent wrinkles from appearing and to keep the skin tight, it is needed to induce the skin to create more collagen or to inject collagen into the skin.

When nutrients required for forming collagen like Vitamin C or peptide are spread or sprayed on the skin, only 0.3% of such nutrients pass through the skin, and the remaining 99.7% remains on the skin, dried and removed away.

Microneedle therapy system (MTS) is used to eliminate such problem. MTS is a device to supply nutrients required to create collagen to the dermis by stimulating the skin or by forming micro channels in the skin using a roller with micro needles projected therefrom.

The thickness of the epidermis is about 0.03 mm to 1 mm. The exposed ends of the micro needles penetrate the epidermis, thereby forming micro channels to the dermis. The nutrients can be effectively supplied to the dermis through the micro channels for sustaining the skin tight or for preventing wrinkles from appearing.

Minute wounds are formed in the skin with the micro needle roller, and the skin is stimulated and creates collagen by natural curing process of the skin, thereby reproducing the skin. That is, collagen is formed by natural curing process and restores the aged skin without hurting the epidermis like laser peeling operation.

Microneedle rollers by prior art have disadvantages that they cannot supply electric stimulation, the roller unit is easy to be disassembled, the microneedles fall off from the roller, and productivity is low since it takes much time to assemble the roller unit.

### SUMMARY OF THE INVENTION

The present invention contrives to solve the disadvantages of the prior art.

An objective of the invention is to provide a microneedle roller that can provide electric stimulation to the skin.

Another objective of the invention is to provide a microneedle roller that has good maintainability and productivity.

In order to achieve the above objectives, the present invention provides an electrically conductive microneedle roller that includes a plurality of discs, a plurality of microneedles, a bracket and a handle.

Each of the discs comprises a first side surface, a second side surface, and an electrically conductive part. The first side surface comprises a plurality of radial grooves. The discs are stacked on one another in a way that the first side surface of one disc contacts the second side surface of the adjacent disc.

The microneedles are received in the radial grooves of the disc, and each of which has a pointed end and a base end. The pointed end protrudes beyond the outer circumference of the disc. The base end is electrically connected to the electrically conductive part of the disc.

The bracket supports the stacked discs. The bracket is electrically conductive. The handle supports the bracket.

The electrically conductive part comprises a circular plate that is provided at the center of the disc.

The handle comprises a electrically conductive wire that is connected to the bracket.

The electrically conductive part of the disc comprises a projection on one side and a recess on the other side. The projection of one electrically conductive part engages with the recess of the adjacent electrically conductive part.

The radial groove comprises an inner end and an outer end. The outer end meets the circumference of the disc. The outer end has a tapered shape that becomes narrower toward the circumference of the disc.

The disc further comprises a recess for receiving bond. The recess for receiving bond surrounds the electrically conductive part of the disc.

The bond is a UV bond.

The disc comprises a disc projection on one side and a disc recess on the other side. The disc projection of one disc engages with the disc recess of the adjacent disc.

The advantages of the present invention are: (1) the microneedle roller provides electric stimulation to the skin and substantially increases skin enhancement effect; (2) the microneedle roller has good productivity since assembly time is reduced; and (3) the service life of the microneedle roller is extended since the microneedles do not fall off from the roller unit.

Although the present invention is briefly summarized, the fuller understanding of the invention can be obtained by the following drawings, detailed description and appended claims.

### BRIEF DESCRIPTION OF THE DRAWINGS

These and other features, aspects and advantages of the present invention will become better understood with reference to the accompanying drawings, wherein:

FIG. 1 is a perspective view of an electrically conductive microneedle roller according to the present invention;

FIG. 2 is an exploded perspective view of a roller unit;

FIG. 3 is a perspective view of a disc;

FIG. 4 is a perspective view of a disc viewed from the other side;

FIG. 5 is an enlarged perspective view of a microneedle, portion A of FIG. 3; and

FIG. 6 is an elevation view of the microneedle.

### DETAILED DESCRIPTION OF THE INVENTION

FIG. 1 shows an electrically conductive microneedle roller 110 that includes a plurality of discs 112, a plurality of microneedles 115, a bracket 130 and a handle 120.

Each of the discs 112 comprises a first side surface 12, a second side surface 14, and an electrically conductive part 1121. The first side surface 12 comprises a plurality of radial grooves 1122. The discs 112 are stacked on one another in a way that the first side surface 12 of one disc 112 contacts the second side surface 14 of the adjacent disc 112.

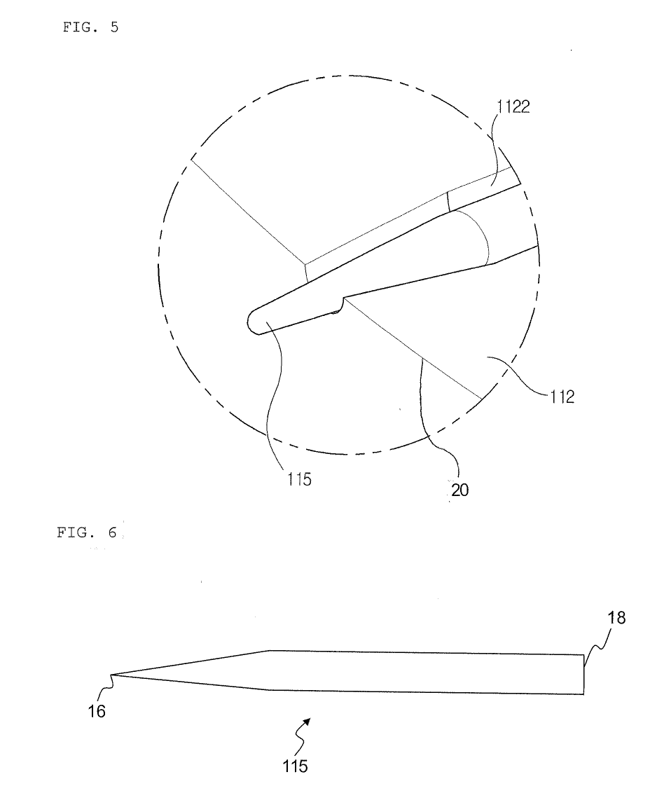

The microneedles 115 are received in the radial grooves 1122 of the disc 112 (refer to FIG. 5), and each of which has a pointed end 16 and a base end 18 (refer to FIG. 6). The pointed end 16 protrudes beyond the outer circumference 20 of the disc. The base end 18 is electrically connected to the electrically conductive part 1121 of the disc 112.

The electrically conductive part 1121 comprises a circular plate that is provided at the center of the disc. The electrically conductive part 1121 of the disc further comprises a projection 1126 on one side and a recess 1127 on the other side. The projection 1126 of one electrically conductive part 1121 engages with the recess 1127 of the adjacent electrically conductive part 1121 thereby combining adjacent electrically conductive parts 1121. The electrically conductive part 1121 is made of copper.

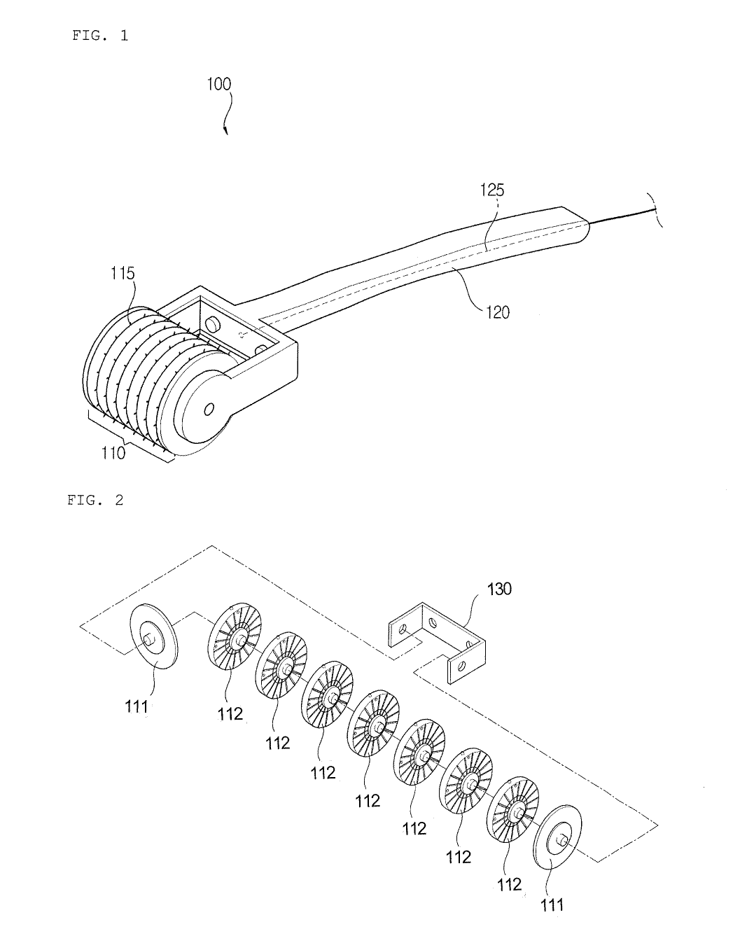

The bracket 130 supports the stacked discs 112 at both ends of the stack. FIG. 2 shows disc covers 111 are positioned at the ends of the stacked discs 112. The bracket 130 is electrically conductive and has a ‘U’ shape. The handle 120 supports the bracket 130. The handle 120 comprises an electrically conductive wire 125 that is connected to the bracket 130. The bracket 130 is connected to the electrically conductive wire 125 and supplies electric current to the electrically conductive parts 1121 of two discs that are positioned at the ends of the stack. The electric current flows to the electrically conductive parts 1121 of the other discs and to the microneedles 115.

Weak electric current stimulation is given to the skin with the microneedles. This causes the skin to contract and to be more elastic thereby enhancing effects of skin care like removing wrinkles.

The disc 112 further comprises a recess 1123 for receiving bond in liquid state. The bond is used to combine the plurality of discs 112. The recess 1123 for receiving bond surrounds the electrically conductive part 1121 of the disc 112. The bond is a UV bond that is cured by ultraviolet ray. The UV bond has a clean bonding surface, and hardens very fast, from 5 to 15 seconds. The use of the UV bond reduces bonding time of the discs 112 thereby increasing the productivity.

The disc 112 comprises one or more disc projections 1125 on one side and one or more disc recesses 1124 on the other side. The disc projections 1125 of one disc 112 engage with the disc recesses 1124 of the adjacent disc 112. The disc projections 1125 and the disc recesses 1124 facilitate assembly of the discs 112 and prevent separate rotation of the discs 112.

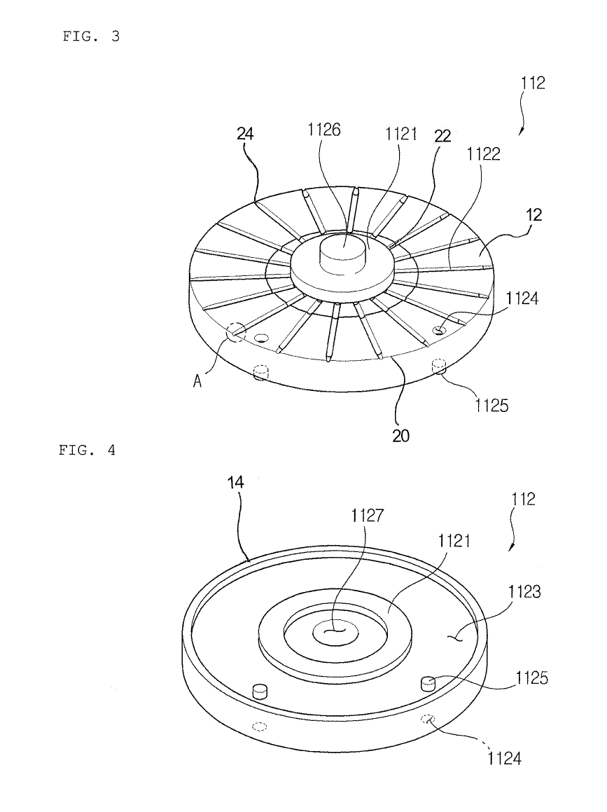

FIG. 5 shows that the microneedle 115 is received in the radial groove 1122 of the disc 112. Referring to FIG. 3, the radial groove 1122 comprises an inner end 22 and an outer end 24. The outer end 24 meets the circumference of the disc 112. The outer end 24 has a tapered shape that becomes narrower toward the circumference 20 of the disc 112. This narrowing shape of the radial groove 1122 confines the microneedle 115 inside the radial groove 1122 and prevents falling off of the microneedle 115 from the disc 112, thereby extending the service life of the microneedle roller 110.

While the invention has been shown and described with reference to different embodiments thereof, it will be appreciated by those skilled in the art that variations in form, detail, compositions and operation may be made without departing from the spirit and scope of the invention as defined by the accompanying claims.

What is claimed is:

1. An electrically conductive microneedle roller comprising:
a) a plurality of discs, each of which comprises a first side surface, a second side surface, and an electrically conductive part, wherein the first side surface comprises a plurality of radial grooves, wherein the discs are stacked on one another in a way that the first side surface of one disc contacts the second side surface of the adjacent disc;
b) a plurality of microneedles that are received in the radial grooves of the disc, and each of which has a pointed end and a base end, wherein the pointed end protrudes beyond the outer circumference of the disc, wherein the base end is electrically connected to the electrically conductive part of the disc;
c) a bracket that supports the stacked discs, wherein the bracket is electrically conductive; and
d) a handle that supports the bracket.

2. The electrically conductive microneedle roller of claim 1, wherein the electrically conductive part comprises a circular plate that is provided at the center of the disc.

3. The electrically conductive microneedle roller of claim 2, wherein the handle comprises a electrically conductive wire that is connected to the bracket.

4. The electrically conductive microneedle roller of claim 2, wherein the electrically conductive part of the disc comprises a projection on one side and a recess on the other side, wherein the projection of one electrically conductive part engages with the recess of the adjacent electrically conductive part.

5. The electrically conductive microneedle roller of claim 2, wherein the radial groove comprises an inner end and an outer end, wherein the outer end meets the circumference of the disc, wherein the outer end has a tapered shape that becomes narrower toward the circumference of the disc.

6. The electrically conductive microneedle roller of claim 2, wherein the disc further comprises a recess for receiving bond, wherein the recess for receiving bond surrounds the electrically conductive part of the disc.

7. The electrically conductive microneedle roller of claim 6, wherein the bond is a UV bond.

8. The electrically conductive microneedle roller of claim 2, wherein the disc comprises a disc projection on one side and a disc recess on the other side, wherein the disc projection of one disc engages with the disc recess of the adjacent disc.
